(19) United States
(12) Patent Application Publication
(10) Pub. No.: US 2021/0145037 A1
(43) Pub. Date: May 20, 2021

(54) PROTEIN HYDROLYSATE FOR SHORT TERM RENAL FUNCTIONING

(71) Applicant: NEWTRICIOUS B.V., Hertogenbosch (NL)

(72) Inventors: Jogchum PLAT, Geulle (NL); Sanne Maria VAN DER MADE, Berlicum (NL); Marcel Alexander BIVERT, Maastricht (NL)

(21) Appl. No.: 17/162,763

(22) Filed: Jan. 29, 2021

Related U.S. Application Data
(63) Continuation of application No. PCT/NL2019/050505, filed on Jul. 30, 2019.

(30) Foreign Application Priority Data
Jul. 31, 2018 (NL) 2021411

(51) Int. Cl. A23L 33/18; A61K 38/01

(52) U.S. Cl. CPC A23L 33/18; A61K 38/012

(57) ABSTRACT

The present invention provides a lysozyme hydrolysate having an effect on renal functioning, as well as food products and food supplements comprising such a hydrolysate.

### CROSS-REFERENCE TO RELATED APPLICATIONS

This application is a continuation of International Application No. PCT/NL2019/050505, filed Jul. 30, 2019, which claims the benefit of and priority to Netherlands Application No. 2021411, filed Jul. 31, 2018, both of which are hereby incorporated by reference herein in their entireties.

### FIELD OF THE INVENTION

The present invention relates to subtilisin A hydrolysate of lysozyme, and food, functional foods and food supplements comprising this in suitable amounts. Food, functional foods, food supplements and the active protein hydrolysates as functional ingredients as such are particularly suited for direct improvement of renal functioning and may be consumed both prophylactically and therapeutically.

### BACKGROUND OF THE INVENTION

Renal failure is a severe condition in which kidneys no longer work properly and can be divided into acute renal failure or chronic renal failure caused by chronic kidney disease (CKD). Chronic kidney disease is a type of kidney disease in which there is gradual loss of kidney function over a period of months or years. Generally at the onset of CDK the subject does not experience symptoms. Still at that moment a decrease in renal functioning especially related to CKD may already be observed through measuring the albumin to creatinine ratio (ACR).

CKD is often a resultant of or related to other conditions such as diabetes, high blood pressure, glomerulonephritis, and polycystic kidney disease. Treatment of CKD generally involves the control of blood pressure, as CKD is often a resultant of high blood pressure. Moreover CKD may result in increased blood pressure so treatment of high blood pressure is also for this reason common. Angiotensin converting enzyme (ACE) inhibitors are commonly used to control blood pressure and may also slow down the progression of CKD. Although the effect of ACE inhibitors on blood pressure may be direct, the effect on renal functioning is long term. Typically the effect on renal functioning is only noticeable over years and mostly only a slowdown of the decrease in renal functioning is induced by ACE inhibitors. Moreover ACE inhibitors are known to have a negative effect on short term renal functioning (Bakris et al., Am. J. of Kidney Diseases, 36(3), 2000, 646-661).

NWT-03 is a subtilisin A hydrolysate of lysozyme suggested for its effect on blood pressure and angiotensin converting enzyme (ACE) inhibition. Plat et al. (British Journal of Nutrition (2017), 117, 942-950) demonstrated an effect of NWT-03 on blood pressure over a period of days in mild-hypertensive human subjects. WO 2006/009448 demonstrated for NWT-03 an ACE inhibiting effect; both in-vitro and in-vivo (in spontaneously hypertensive rats (SHR)). Moreover, an effect on blood pressure was found in the same model (SHR). NWT-03 has also been suggested to have an effect on long term renal functioning in hypertensive ZDF rats that are obese and have type-2 diabetes, probably through its ACE inhibiting effect (Wang et al., Plos one, 2012; 7(10), e46781).

Chronic kidney disease or in general kidney damage may be diagnosed through measuring the albumin to creatinin Ratio (ACR) in the urine. A healthy kidney does not leak albumin to urine. An alternative method for establishing kidney damage is to measure the glomerular filtration rate (GFR), for which is a blood test is needed.

### SUMMARY OF THE INVENTION

Proper renal functioning is important for a subject's health. At onset, the subject may not be aware of deteriorated renal functioning, however, deteriorated renal functioning is in general progressive and leads to further and more severe symptoms and conditions. Ultimately, poor renal functioning may lead to an early death. NWT-03 is known for its effect on ACE inhibition and reducing blood pressure in hypertensive subjects. Blood pressure management in subjects with deteriorated renal functioning is known to be effective for long term slowdown of deterioration of renal functioning. Blood pressure management implies that the blood pressure of subjects with raised blood pressure is lowered and simultaneously the deteriorating of renal functioning is slowed down.

The inventors now surprisingly found in a randomized double-blind, placebo controlled, cross-over study that a blood pressure independent direct improvement of renal function may be obtained by a nutritional composition comprising an effective amount of subtilisin A hydrolysate of lysozyme and/or that a direct deterioration in renal functioning may be prevented, wherein the deterioration in renal functioning is not related to a change in blood pressure. Such direct improvement was not to be expected of a subtilisin A hydrolysate of lysozyme as improvements for renal functioning of ACE inhibitors are long term and related to a change in blood pressure. Especially since ACE inhibitors are commonly associated with short term deterioration of renal functioning, the effect the inventors found is beyond expectation.

### DETAILED DESCRIPTION OF THE INVENTION

The invention thus concerns a method for blood pressure independent direct improvement of renal functioning in a subject, wherein a nutritional composition comprising subtilisin A hydrolysate of lysozyme is administered to a subject. In particular an effective amount of subtilisin A hydrolysate of lysozyme is administered to the subject. Preferably the subject is in need of direct improvement of renal functioning.

The invention can also be worded as the use of subtilisin A hydrolysate of lysozyme or a composition comprising a subtilisin A hydrolysate of lysozyme for the manufacture of a nutritional composition for blood pressure independent direct improvement of renal functioning in a subject.

The invention can also be worded as a nutritional composition comprising subtilisin A hydrolysate of lysozyme for use in blood pressure independent direct improvement of renal functioning in a subject.

The invention also concerns a method for preventing a direct deterioration in renal functioning in a subject, wherein a nutritional composition comprising subtilisin A hydrolysate of lysozyme is administered to a subject and wherein the deterioration in renal functioning is not related to a change in blood pressure. In particular an effective amount of subtilisin A hydrolysate of lysozyme is administered to the subject. Preferably the subject is in need of preventing a direct deterioration in renal functioning.

The invention can also be worded as the use of a composition comprising subtilisin A hydrolysate of lysozyme for the manufacture of a nutritional composition for preventing a direct deterioration in renal functioning in a subject, wherein the deterioration in renal functioning is not related to a change in blood pressure.

The invention can also be worded as a nutritional composition comprising subtilisin A hydrolysate of lysozyme for use in preventing a direct deterioration in renal functioning in a subject, wherein the deterioration in renal functioning is not related to a change in blood pressure.

Furthermore the invention concerns a method for blood pressure independent prevention of direct deterioration of renal functioning in subjects diagnosed with metabolic syndrome, wherein a nutritional composition comprising subtilisin A hydrolysate of lysozyme is administered to a subject. In particular an effective amount of subtilisin A hydrolysate of lysozyme is administered to the subject. Preferably the subject is in need of prevention of direct deterioration of renal functioning.

The invention can also be worded as the use of a composition comprising subtilisin A hydrolysate of lysozyme for the manufacture of a nutritional composition for blood pressure independent prevention of direct deterioration of renal functioning in subjects diagnosed with metabolic syndrome.

The invention can also be worded as a nutritional composition comprising subtilisin A hydrolysate of lysozyme for use in blood pressure independent prevention of direct deterioration of renal functioning in subjects diagnosed with metabolic syndrome.

The invention also concerns a method for direct improvement of renal functioning in a subject not suffering from hypertension, wherein a nutritional composition comprising subtilisin A hydrolysate of lysozyme is administered to a subject. In particular an effective amount of subtilisin A hydrolysate of lysozyme is administered to the subject. Preferably the subject not suffering from hypertension and is in need of direct improvement of renal functioning.

The invention can also be worded as the use of subtilisin A hydrolysate of lysozyme or a composition comprising a subtilisin A hydrolysate of lysozyme for the manufacture of a nutritional composition for direct improvement of renal functioning in a subject not suffering from hypertension.

The invention can also be worded as a nutritional composition comprising subtilisin A hydrolysate of lysozyme for use in direct improvement of renal functioning in a subject not suffering from hypertension.

The invention also concerns a method for preventing a direct deterioration in renal functioning in a subject not suffering from hypertension, wherein a nutritional composition comprising subtilisin A hydrolysate of lysozyme is administered to a subject. In particular an effective amount of subtilisin A hydrolysate of lysozyme is administered to the subject. Preferably the subject not suffering from hypertension is in need of preventing a direct deterioration in renal functioning.

The invention can also be worded as the use of a composition comprising subtilisin A hydrolysate of lysozyme for the manufacture of a nutritional composition for preventing a direct deterioration in renal functioning in a subject not suffering from hypertension.

The invention can also be worded as a nutritional composition comprising subtilisin A hydrolysate of lysozyme for use in preventing a direct deterioration in renal functioning in a subject not suffering from hypertension.

### General Definitions

The term “comprising” is to be interpreted as specifying the presence of the stated parts, steps or components, but does not exclude the presence of one or more additional parts, steps or components. A peptide sequence comprising region X, may thus comprise additional regions, i.e. region X may be embedded in a larger peptide region.

The term “food” refers herein to liquid, semi-liquid or solid food products suitable for human and/or animal consumption.

“Functional food” refers to a food product which comprises one or more active ingredients, especially with subtilisin A hydrolysate of lysozyme according to the invention as active ingredient, whereby for the latter the active ingredient improves renal functioning in a subject or prevents a direct deterioration in renal functioning in a subject.

“Food supplement” refers to supplements suitable for human and/or animal consumption which comprise a suitable amount of one or more subtilisin A hydrolysate of lysozyme according to the invention as functional ingredient. Supplements may be in the form of pills, sachets, powders and the like.

“Subjects” means any member of the class mammals, including without limitation humans, non-human primates, farm animals, domestic animals and laboratory animals.

“Eggs” refer herein preferably to chicken eggs, although eggs from other birds may also be used.

“Egg protein hydrolysates” is used herein as a general term to refer to protein hydrolysates (prepared in vitro) of whole eggs, egg fractions (e.g. egg white or egg yolk) or of substantially pure egg proteins, especially lysozyme.

“Lysozyme hydrolysate” is used herein as a general term to refer to hydrolysates, for example prepared in vitro, of lysozyme.

“Non-hydrolysed egg protein” or “undigested egg protein” is used herein as a general term to refer to whole eggs, egg fractions (e.g. egg white or egg yolk), or substantially pure egg proteins, especially lysozyme, which have not been hydrolysed in vitro.

“Metabolic Syndrome” refers to multiple interrelated clinical disorders, including obesity, insulin resistance and hyperinsulinemia, glucose intolerance, hypertension and dyslipidemia (hypertriglyceridemia and low HDL cholesterol levels) as described e.g. in Moller and Kaufman (Annual Rev. of Medicine 2005, vol 56:45-62). In the context of the current invention, preferably metabolic syndrome is diagnosed by an impaired glucose regulation/insulin resistance and ≥2 other criteria from 1) Impaired glucose regulation/insulin resistance 2) abdominal obesity 3) hypertriglyceridemia 4) low levels of HDL cholesterol 5) microalbuminuria, wherein the clinical features are further defined according to the WHO definitions as described in Moller and Kaufman.

“Degree of hydrolysis” is the proportion of cleaved peptide bonds in a protein hydrolysate.

### Subject

In one embodiment of the invention preferably the subject is human. Preferably the subject is diagnosed with one or more of diabetes type 1, diabetes type 2, obesity, glomerulonephritis and metabolic syndrome, more preferably metabolic syndrome. A subject diagnosed with one or more of diabetes type 1, diabetes type 2, obesity, glomerulonephritis and metabolic syndrome is particularly in need of the composition, as such subject is at risk of decreased renal functioning. Preferably the subject is diagnosed with chronic kidney disease (CKD). A subject diagnosed with CKD is particularly in need of the composition, as such subject is suffering from decreased renal functioning. Preferably the subject is in need of blood pressure independent direct improvement of renal functioning, preferably the subject is in need of prevention of direct deterioration of renal functioning, preferably the subject is diagnosed with metabolic syndrome and in need prevention of direct deterioration of renal functioning in subjects, preferably the subject is not suffering from hypertension and in need of direct improvement of renal functioning, preferably the subject is not suffering from hypertension and in need of preventing a direct deterioration in renal functioning. In one embodiment the subject is not diagnosed with diabetes type 2.

### Renal Functioning

Albumin is a protein that is present in large amounts in the blood. When the kidneys are working properly, only tiny amounts of albumin pass from the bloodstream into the urine. In kidney failure, the last stage of a slow process of decline in kidney function, large amounts of protein leak into the urine. Well before occurrence of such extensive damage, small changes in the blood-filtering parts of the kidney allow very small, but abnormal amounts of albumin to leak through. Urinary albumin increases when the kidney leaks small amounts of albumin into the urine, which is a consequence of abnormally high permeability for albumin in the glomerulus. Generally, subjects do not show any other symptoms at the moment small amounts of albumin leak through.

For determining renal functioning the albumin-to-creatinine ratio (ACR) in the urine is used. It is known in the art how to measure the ACR. Renal function is normal when no measurable amount of albumin leaks through and the albumin-to-creatinine ratio in the urine cannot be determined. Deterioration of renal functioning is defined as an increase of the ACR; improvement of renal functioning is defined as a decrease of the ACR.

### Blood Pressure

Blood pressure and blood pressure measurement are known in the art. Blood pressure is characterized by the systolic blood pressure (SBP) and the diastolic blood pressure (DBP). A normal blood pressure for a human adult is about 120 mmHg (SBP)/80 mmHg (DBP). Raised or high blood pressure can be divided into different levels, such as Mild (140-160/95-104), Moderate 140-180/105-114) and Severe (160+/115+). “Blood pressure” refers to the blood pressure of a subject, characterized by the systolic blood pressure (SBP) and/or the diastolic blood pressure (DBP) of this subject. Hypertension is the same as raised of high blood pressure, so a subject suffering from hypertension suffers from a raised or high blood pressure.

The inventors surprisingly found that the renal functioning in a subject can be directly improved or a direct deterioration in renal functioning in a subject can be prevented and that these effects cannot be the result of an improvement in blood pressure. Independent of blood pressure is the same as not related to a change in blood pressure and/or not caused by a change in blood pressure. Thus in the context of the present invention, ‘blood pressure independent’ means that the direct improvement of renal functioning in a subject and/or the prevention of direct deterioration of renal functioning in a subject is not related to, triggered by and/or caused by a change in blood pressure. In particular ‘blood pressure independent’ means that the direct improvement of renal functioning in a subject and/or the prevention of direct deterioration of renal functioning in a subject is not related to and/or triggered by and/or caused by a lowering of in blood pressure. As an example, the renal functioning in a subject directly improves or a direct deterioration of renal functioning in a subject is prevented whilst the blood pressure does not decrease.

In one embodiment according to the present invention, the blood pressure, more in particular the systolic blood pressure and/or the diastolic blood pressure does not substantially change relative to the blood pressure at the moment of first administration or intake of the present nutritional composition comprising subtilisin A hydrolysate of lysozyme. Preferably the blood pressure does not substantially change within the time period the direct improvement or the prevention of direct deterioration of renal functioning in a subject occurs. Preferably the systolic blood pressure and/or the diastolic blood pressure does not change by more than 20% relative to the blood pressure at the moment of first administration or intake of the present nutritional composition comprising subtilisin A hydrolysate of lysozyme, preferably does not change by more than 10%, most preferably does not change by more than 5%. Preferably the systolic blood pressure and/or the diastolic blood pressure does not change by more than 20% relative to the blood pressure at the moment of first administration or intake of the present nutritional composition comprising subtilisin A hydrolysate of lysozyme, preferably does not change by more than 10%, most preferably does not change by more than 5% within the time period the direct improvement or the prevention of direct deterioration of renal functioning in a subject occurs. Preferably the systolic blood pressure and/or the diastolic blood pressure does not decrease by more than 20% relative to the blood pressure at the moment of first administration or intake of the present nutritional composition comprising subtilisin A hydrolysate of lysozyme, preferably does not decrease by more than 10%, most preferably does not decrease by more than 5%. Preferably the systolic blood pressure and/or the diastolic blood pressure does not decrease by more than 20% relative to the blood pressure at the moment of first administration or intake of the present nutritional composition comprising subtilisin A hydrolysate of lysozyme, preferably does not change by more than 10%, most preferably does not change by more than 5% within the time period the direct improvement or the prevention of direct deterioration of renal functioning in a subject occurs.

### Direct Improvement

The inventors observed the renal functioning in a subject can be improved directly and/or a deterioration in renal functioning in a subject can be prevented directly. Within the present context, ‘directly’ or ‘direct’ means that the observed occurs within a specific time period after first administration or intake of the nutritional composition according to the invention. In an embodiment of the present invention, ‘direct’ is preferably within 12 weeks of first administration or intake of the nutritional composition, more preferably within 8 weeks, even more preferably within 4 weeks, most preferably within 2 weeks of first administration or intake of the nutritional composition according to the invention.

In the present context ‘long term’ means that an effect can only be observed after a specific time period after first administration of a nutritional composition to a subject or after first intake of the nutritional composition by the subject. A long term effect is defined as an effect that is observable only after 8 weeks of first administration or intake of a nutritional composition, more preferably after 12 weeks, even more preferably after 16 weeks, most preferably after 20 weeks.

### Enzyme

According to the present invention, the nutritional composition comprises subtilisin A hydrolysate of lysozyme. Thus lysozyme is hydrolysed with subtilisin A. Subtilisin A is an endopeptidase, more specifically a serine protease even more specifically a subtilase. Subtilisin A is amongst others commercially marketed as Alcalase™.

### Hydrolysate of Lysozyme

It is known in the art how to obtain a subtilisin A hydrolysate of lysozyme. A method for obtaining a subtilisin A hydrolysate of lysozyme is for example described in WO 2006/009448 and example 1.

Lysozyme is commercially available and may for example be obtained from BouwhuisEnthoven (100% pure protein). Lysozyme may also be extracted from eggs and subsequently purified. In one embodiment of the invention, the lysozyme that is subjected to subtilisin A hydrolysis is preferably purified lysozyme. The concentration of lysozyme in egg white is high (3-4%) as compared to other sources of lysozyme. A process used routinely for lysozyme purification is cation exchange chromatography. Lysozyme is bound to a cation exchanger at the pH as is (pH 9). This may be done in a stirred tank reactor or in a chromatography column. After elution from the adsorption particles by salt, lysozyme is pure enough for food applications. Anion exchange chromatography at pH 4 may be applied for further purification in order to obtain highly pure lysozyme, suitable for pharmaceutical applications. The advantages of this purification process are that the starting material (egg white) is not altered, the process can easily be up-scaled, for food applications just one adsorption process is needed and the biological activity is retained. Thus, in one embodiment according to the present invention, the lysozyme that is subjected to subtilisin A hydrolysis is purified using cation exchange chromatography.

The subtilisin A hydrolysate of lysozyme according to the invention preferably comprises a high proportion of di- and/or tri peptides, preferably at least 10, 20, 30, 40, 50, 60, 70, wt. % or more of the weight of subtilisin A hydrolysate of lysozyme, more preferably at least 20 wt. %, even more preferably at least 30 wt. %, most preferably at least 40 wt. %. In one embodiment peptides of longer chain length and higher molecular weight, such as peptides with 4, 5, 6, 7, 8 or more amino acids, are therefore preferably present in amounts of less than 60 wt. % of the weight of subtilisin A hydrolysate of lysozyme.

In one embodiment, the subtilisin A hydrolysate of lysozyme according to the invention preferably comprises peptides of less than 0.5 kD in a proportion of at least 25, 30, 40 or 45 wt. % of the weight of subtilisin A hydrolysate of lysozyme, preferably the subtilisin A hydrolysate of lysozyme comprises at least 30 wt. %, most preferably at least 40 wt. %. peptides of less than 0.5 kD. Preferably the subtilisin A hydrolysate of lysozyme according to the invention preferably comprises peptides of less than 0.5 kD in a proportion of at most 80, 70, 60 or 50 wt. % of the weight of subtilisin A hydrolysate of lysozyme.

In one embodiment, the subtilisin A hydrolysate of lysozyme according to the invention comprises a distribution of fragments of molecular weight of <0.5 kD:from 0.5 to 1.0 kD:>1 kD) in a ratio of 30-70:70-15:70-15. As an indication, fragments of less than 0.5 kD correspond to fragments of less than 4-5 amino acids, while fragments of about 0.5-1.0 kD correspond to 4-9 amino acids and fragments of above 1 kD correspond to peptide fragments of above 9 amino acids.

Preferably, the degree of hydrolysis of the subtilisin A hydrolysate of lysozyme is at least 15%, more preferably at least 20%, most preferably at least 30%. The degree of hydrolysis is preferably determined with the TNBS method, as for example described in WO 2006/009448. The degree of hydrolysis is the difference in the number of free amino groups in the hydrolysate and the number of free amino groups in the intact protein divided by the total amount of peptide bonds in the hydrolysate/intact protein. Generally the degree of hydrolysis is expressed in percentage, so the resultant value is multiplied by 100%.

A molecular weight distribution of the subtilisin A hydrolysate of lysozyme preferably comprises 30-70% target peptides of less than 0.5 kD and 15-70% peptides between 0.5 and 1 kD based of the weight of subtilisin A hydrolysate of lysozyme. The molecular weight distribution, peptide chain length distribution and maximum peptide weight of the hydrolysate can be determined by methods known in the art, such as SDS-PAGE analysis, HPLC analysis, MALDI-TOF (Matrix-Assisted Laser Desorption/Ionisation Time-of-Flight mass spectrometry, as described by Kaufmann, J. Biotechn 1995, 41:155-175 and Soeryapranata et al. 2002, J. Food Sci. 67:534-538), HP-GPC (high performance gel permeation chromatrography, as described in Terheggen-Lagro et al. BMC Pediatrics 2002, 2:10) and Edman degradation (Siemensma et al. Trends Food Sci Technology 1993, 4:16-21). The maximum molecular peptide weight of the target protein is preferably less than 10 kD.

Alternatively a composition resembling a subtilisin A hydrolysate of lysozyme of this invention can be made de novo making use of chemical synthesis methods. In particular a di- and a tri-peptide library can be made by combinatorial chemistry thereby forming all possible combinations of dipeptides and tripeptides. From this pool, or library of di- and tripeptides a mixture can be composed having essentially the same activity on renal functioning as the hydrolysates described above.

### Effective Amount

The nutritional compositions comprising subtilisin A hydrolysate of lysozyme according to the invention preferably directly improve renal functioning or prevent a direct deterioration after regular, preferably daily, intake of an effective amount. An effective amount of the nutritional composition or effective dose in the context of the current invention is such an amount of the claimed nutritional composition to obtain the the claimed therapeutic or prophylactic effect in vivo. The effective amount which needs to be added depends on a number of factors, such as the subject (e.g. human or animal), the dosage form (daily, several times a day, weekly) and product composition and/or texture. It is within the realm of a skilled person to determine the effective amount using routine experimentation. Preferably an effective amount is at least 0.1 grams of the claimed composition per day, more preferably at least 0.5 grams, even more preferably at least 1 grams, even more preferably at least 2 grams, most preferably at least 4 grams. Preferably an effective amount is at least 7 mg/kg body weight of the subject per day, more preferably at least 14 mg/kg, even more preferably at least 28 mg/kg, most preferably at least 56 mg/kg body weight of the subject per day.

### Nutritional Composition

Provided are thus nutritional compositions comprising the subtilisin A hydrolysates of lysozyme, generated as described above and optionally further enriched and/or purified for use according to the present invention. Also provided are nutritional compositions, especially food product, functional food product and/or food supplement compositions or in other words food products, functional food products and/or food supplements comprising a suitable amount of a subtilisin A hydrolysates of lysozyme for use according to the present invention.

Food supplements and food products comprising at least one subtilisin A hydrolysate of lysozyme according to the invention may be made as known in the art. Food supplements may for example be in the form of any dosage form such as tablets, pills, powder sachets, gels, capsules, and the like. Intake by the subject is preferably oral. Food products may be in the form of drinks, solid or semi-solid foods, such as snacks, deserts, sauces, whole meals, etc. Preferably the food product is a product which is consumed on a regular basis, preferably daily, such as staple foods, e.g. bread, noodles, soft drinks, dairy products such as cheese, yoghurt, fermented dairy products, milk, butter etc. The subtilisin A hydrolysates of lysozyme may thus be added to a food base or may be incorporated into the food product during its production process. Thus, any existing food or food supplement products comprising a subtilisin A hydrolysates of lysozyme for use according to the present invention are included herein.

In a preferred embodiment the food product is a drink, preferably based on a fruit juice or vegetable juice, although milk based drinks are also included. The drink may be made in daily dosage volumes of 50 ml, 100 ml, 150 ml, 200 ml or more. It is understood that the food supplement or food product may further comprise additional food-grade ingredients, such as but not limited to flavourings, vitamins, minerals, stabilizers, emulsifiers, other biologically active ingredients, food bases/nutrients such as a protein, carbohydrate and/or fat component, and the like. The subtilisin A hydrolysate of lysozyme may be added at any stage during the normal production process of the food product/food supplement.

The food product/supplement may also comprise other inactive ingredients and carriers, such as e.g. glucose, lactose, sucrose, mannitol, starch, cellulose or cellulose derivatives, e.g. carboxymethylcellulose (CMC), magnesium stearate, stearic acid, sodium saccharin, talcum, magnesium carbonate and the like. It may also comprise water, electrolytes, essential and non-essential amino acids, trace elements, minerals, fibre, sweeteners, flavourings, colorants, emulsifiers and stabilizers (such as soy lecithin, citric acid, esters of mono- or di-glycerides), preservatives, binders, fragrances, and the like.

For food supplements in the form of pills or capsules, a coating may be added which changes the place and/or time of release in vivo of the hydrolysate. Slow release formulations are known in the art.

Alternatively, the product may be ingested prophylactically by subjects at risk of developing deteriorated renal functioning. In one embodiment, the composition according to the invention is an aqueous composition. The composition according to the invention may be a ready-for-use solution, as a stock solution from which the daily dose may be obtained or prepared by dilution or it may be a dry composition from which a daily dose may be obtained by adding a fluid. The composition may also be used as dry matter and mixed with a food stuff, The skilled person is well aware of these and other ways of administering a composition to a subject in need of the composition.

Preferably, the nutritional composition is ingested either as a food supplement, e.g. in the form of tablets, sachets, etc., or as a functional food, e.g. in the form of drinks, semi-solid or solid food products.

### EXAMPLES

### Example 1: Preparation of a Subtilisin a Hydrolysate of Lysozyme

A 5% (w/v) solution of lysozyme in water (100% protein content, BouwhuisEnthoven, Raalte, The Netherlands) was prepared and adjusted to a pH between 7.5 and 8.5 with 3 M KOH. Hydrolysis was started by adding Alcalase™ (Novozymes) to a final concentration of 4% on protein basis. The solution was incubated for a total of 6 hours at 60° C., under continuous stirring. Alcalase™ was then inactivated by increasing the temperature to 90° C. for 15 minutes. The solution was then cooled down to 2° C. and stored overnight under continuous stirring.

The resulting hydrolysate solution was filtered through a 10 μm filter and subsequently through a 1 μm filter. Thereafter, the filtrate was heat treated for 15 s at 135° C. and concentrated to a dry matter of 57° Brix (approximately dry matter of 45%) by a NIRO evaporator at a flow of 3300 L/h at 90° C. After evaporation, the product was spray dried to obtain a powder with very good flowability properties, as evidenced by visual observation.

The final product had the following characteristics: white powder, good solubility, degree of hydrolysis of 21% (TNBS method) and a maximum molecular weight of less than 10 kDa. Peptide size distribution was as follows: 46%<500 Da, 23% 500-1000 Da, 32%>1000 Da. This product is herein further referred to as NWT-03.

### Example 2: Clinical Study

The study concerned a randomized, double-blind, placebo controlled, cross-over study. The objective of the study is towards the 4-weeks effect of NWT-03 on urinary albumin-to-creatinine ratio and blood pressure. Urine samples to determine albumin and creatinine concentrations were collected at the start and the end of each 4-week intervention period. Office blood pressure measurements were performed at screening, and at days 0, 2 and 27 of each intervention period. Four blood pressure recordings were made with 1 minute intervals after the subject had rested for 5 minutes. The average of the last three readings was noted for study purposes. All measurements were performed in otherwise healthy subjects yet meeting the criteria to be diagnosed with metabolic syndrome. Each intervention period was separated by a wash-out period of typically four weeks, but it was allowed to extend or shorten this period with maximally 2 weeks. NWT-03 was provided in sachets as a white powder that had good solubility. Subjects were asked to dissolve the powder in approximately 200 mL of water prior to consumption. Placebo was also provided as a powder that was similar to NWT-03 in colour and taste. 79 subjects were randomized at the start of the study. Albumin-to-creatinine ratio (ACR) could be measured on all time points in 18 subjects. A significant difference in the change in albumin-to-creatinine ratio (ACR) was found between control and NWT-03 group (see Table 1). ACR decreased in the NWT-03 group, while an increase was observed in the control group. Both systolic and diastolic blood pressure did not change during the four-week study period. The blood pressure was of the subjects was normal, not raised.

TABLE 1
Baseline values and changes in Albumin:Creatinin Ratio
(ACR), Systolic Blood Pressure (SBP) and Diastolic
Blood Pressure (DBP) in the control and NWT-03 group.

| | | Control | NWT-03 |
|---|---|---|---|
| ACR (g/molCre) | Baseline | 0.69 | 0.82 |
| | Change (27 days) | 0.34 | −0.08 |
| | Change (%) | +49% | −10% |
| | P_{interaction} | 0.005 | |
| SBP (mmHg) | Baseline | 130 | 131 |
| | Change (27 days) | 0 | −2 |
| | Change (%) | 0 | −2% |
| | P_{interaction} | NS | |
| DBP (mmHg) | Baseline | 85 | 86 |
| | Change (27 days) | −1 | −3 |
| | Change (%) | −1% | −3% |
| | P_{interaction} | NS | |

NS: not significant

1. A method of direct improvement of blood pressure-independent renal functioning in a subject, comprising administering to a subject in need thereof a nutritional composition comprising subtilisin A hydrolysate of lysozyme, wherein the direct improvement occurs within 8 weeks after first administration of the nutritional composition.

2. The nutritional composition for use according to claim 1, wherein the subject is not suffering from hypertension.

3. The method according to claim 1, wherein at least 30 wt. % of the subtilisin A hydrolysate of lysozyme are peptides of lysozyme that have a molecular weight of less than 0.5 kD.

4. The method according to claim 1, wherein at least 10 wt. % of the subtilisin A hydrolysate of lysozyme are di- and/or tripeptides.

5. The method according to claim 1, wherein systolic blood pressure and/or the diastolic blood pressure does not change by more than 10% relative to the blood pressure at the moment of first administration.

6. The method according to claim 1, wherein the subject is diagnosed with metabolic syndrome.

7. The method according to claim 1, wherein the subject is diagnosed with chronic kidney disease.

8. The method according to claim 1, wherein the nutritional composition is one of a food supplement or a food product.

9. A method of preventing direct deterioration in renal functioning in a subject, comprising administering to a subject in need thereof a nutritional composition comprising subtilisin A hydrolysate of lysozyme, wherein the deterioration in renal functioning is not related to a change in blood pressure and wherein the prevention of direct deterioration occurs within 8 weeks after first administration of the nutritional composition.

10. The nutritional composition for use according to claim 1, wherein the subject is not suffering from hypertension.

11. The method according to claim 1, wherein at least 30 wt. % of the subtilisin A hydrolysate of lysozyme are peptides of lysozyme that have a molecular weight of less than 0.5 kD.

12. The method according to claim 1, wherein at least 10 wt. % of the subtilisin A hydrolysate of lysozyme are di- and/or tripeptides.

13. The method according to claim 1, wherein the subject is diagnosed with metabolic syndrome.

14. The method according to claim 1, wherein the subject is diagnosed with chronic kidney disease.

15. The method according to claim 1, wherein the nutritional composition is one of a food supplement or a food product.

16. A method of direct improvement of renal functioning in a subject not suffering from hypertension, the method comprising administering to the subject a nutritional composition comprising subtilisin A hydrolysate of lysozyme, wherein the direct improvement occurs within 8 weeks after first administration of the nutritional composition.

17. The method according to claim 1, wherein at least 30 wt. % of the subtilisin A hydrolysate of lysozyme are peptides of lysozyme that have a molecular weight of less than 0.5 kD.

18. The method according to claim 1, wherein at least 10 wt. % of the subtilisin A hydrolysate of lysozyme are di- and/or tripeptides.

19. The method according to claim 1, wherein the subject is diagnosed with metabolic syndrome.

20. The method according to claim 1, wherein the subject is diagnosed with chronic kidney disease.

21. The method according to claim 1, wherein the nutritional composition is one of a food supplement or a food product.

22. A method of blood pressure-independent prevention of direct deterioration of renal functioning in subjects diagnosed with metabolic syndrome, comprising administering to the subject a nutritional composition comprising subtilisin A hydrolysate of lysozyme.
